(19) United States
(12) Patent Application Publication
(10) Pub. No.: US 2006/0166190 A1
(43) Pub. Date: Jul. 27, 2006

(54) Magnetism based nucleic acid amplification

(76) Inventors: Xin Xie, Beijing (CN); Xu Zhang, Beijing (CN); Depu Chen, Beijing (CN); Weiyang Fei, Beijing (CN); Jing Chen, Beijing (CN)

Correspondence Address: MORRISON & FOERSTER LLP, 12531 HIGH BLUFF DRIVE, SUITE 100, SAN DIEGO, CA, 92130-2040, US

(21) Appl. No.: 10/538,443

(22) PCT Filed: Dec. 31, 2002
(86) PCT No.: PCT/CN02/00940

§ 371 Date: Oct. 14, 2005

(30) Foreign Application Priority Data
Dec. 10, 2002 (CN) 02155237.1

(51) Int. Cl. C12Q 1/70; C12Q 1/68

(52) U.S. Cl. 435/5; 435/6

(57) ABSTRACT

This invention relates generally to the field of nucleic acid amplification. In particular, the invention provides processes and kits for amplifying a nucleic acid of a target cell or virus using, using inter alia, binding between a target cell, cellular organelle or virus with a magnetic microbead.

### TECHNICAL FIELD

This invention relates generally to the field of nucleic acid amplification. In particular, the invention provides processes and kits for amplifying a nucleic acid of a target cell or virus using, using inter alia, binding between a target cell, cellular organelle or virus with a magnetic microbead.

### BACKGROUND ART

As a basic technique for diagnostic analysis and molecular biology research, the invention of various nucleic acid amplification procedures, e.g., PCR, promotes the development of life sciences. Complicated and time-consuming, however, the preparation of PCR templates is often the rate-limiting step. How to realize the rapid and simple preparation of PCR templates? It is a promising method for the template preparation to produce automatic mini bio-chip. The build up of the PCR chips is developing progressively. It is suitable for the automation of the analytical process to integrate the templates preparation and PCR process. Thus the lab-on-a-chip system can be built up for biochemical analysis.

For this purpose, this invention adopts the magnetic micro-beads which can be easily operated on the electromagnetic chips as the carrier for the adsorption of separated cells and nucleic acids. The adsorbed cells and nucleic acids can be used as template in various nucleic acid amplification, e.g., PCR, without elution. The magnetic micro-beads have insignificant influence on the specificity and efficiency of nucleic acid amplification. This invention integrates the cell separation, nucleic acid preparation and nucleic acid amplification on the magnetic micro-beads and is useful in the build-up of a biochip and micro-fluid system.

### DISCLOSURE OF THE INVENTION

In one aspect, the present invention is directed to a process for amplifying a nucleic acid of a target cell or virus, which process comprises: a) contacting a sample containing or suspected of containing a target cell or virus with a magnetic microbead; b) allowing said target cell or virus, if present in said sample, to bind to said magnetic microbead to form a conjugate between said target cell or virus and said magnetic microbead; and c) separating said conjugate from other undesirable constituents via a magnetic force to isolate said target cell or virus from said sample; and d) applying said separated conjugate to a nucleic acid amplification system to amplify a nucleic acid from said target cell or virus.

In another aspect, the present invention is directed to a kit for amplifying a nucleic acid of a target cell or virus, which kit comprises in a same or different container(s): a) a magnetic microbead for contacting a sample containing or suspected of containing a target cell or virus; b) means for allowing said target cell or virus, if present in said sample, to bind to said magnetic microbead to form a conjugate between said target cell or virus and said magnetic microbead; c) means for separating said conjugate from other undesirable constituents via a magnetic force from said sample; and d) a nucleic acid amplification system to amplify a nucleic acid from said target cell or virus.

In still another aspect, the present invention is directed to a process for amplifying a nucleic acid of a target cell or virus, which process comprises: a) contacting a sample containing or suspected of containing a target cell or virus with a magnetic microbead; b) allowing said target cell or virus, if present in said sample, to bind to said magnetic microbead to form a conjugate between said target cell or virus and said magnetic microbead; and c) separating said conjugate from other undesirable constituents via a magnetic force to isolate said target cell or virus from said sample; d) releasing a nucleic acid from said cell-microbead or virus-microbead conjugate to form a nucleic acid-microbead conjugate; and d) applying said nucleic acid-microbead conjugate to a nucleic acid amplification system to amplify said nucleic acid from said target cell or virus.

In yet another aspect, the present invention is directed to a kit for amplifying a nucleic acid of a target cell or virus, which kit comprises in a same or different container(s): a) a magnetic microbead for contacting a sample containing or suspected of containing a target cell or virus; b) means for allowing said target cell or virus, if present in said sample, to bind to said magnetic microbead to form a conjugate between said target cell or virus and said magnetic microbead; c) means for separating said conjugate from other undesirable constituents via a magnetic force from said sample; d) means for releasing a nucleic acid from said cell-microbead or virus-microbead conjugate to form a nucleic acid-microbead conjugate; and e) a nucleic acid amplification system to amplify a nucleic acid from said target cell or virus.

### BRIEF DESCRIPTION OF THE DRAWINGS

FIG. 1 illustrates PCR products of the HLA-A allele gene (1,100 bp). The positive control is PCR product from DNA isolated using conventional method. Three (3) μl of sample were applied to the gel. Lanes are (M): DNA mass ladder (DL-2000, TaKaRa, Japan); (1): negative control; (2): positive control; (3, 4): the “Microbead-PCR” product with templates prepared from whole blood sample by our protocol; (5, 6): the “Microbead-PCR” products with templates prepared from saliva sample by our protocol; (7, 8): 2 μl of whole blood added as templates; and (9, 10): 2 μl of saliva added as templates.

### MODES OF CARRYING OUT THE INVENTION

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “specific binding” refers to the binding of one material to another in a manner dependent upon the presence of a particular molecular structure. For example, a receptor will selectively bind ligands that contain the chemical structures complementary to the ligand binding site(s).

As used herein, “specific binding pair” refers to any substance, or class of substances, which has a specific binding affinity for the ligand to the exclusion of other substances. In one embodiment, the specific binding pair includes specific binding assay reagents which interact with the sample ligand or the binding capacity of the sample for the ligand in an immunochemical manner. For example, there will be an antigen-antibody or hapten-antibody relationship between reagents and/or the sample ligand or the binding capacity of the sample for the ligand. Additionally, it is well understood in the art that other binding interactions between the ligand and the binding partner serve as the basis of specific binding assays, including the binding interactions between hormones, vitamins, metabolites, and pharmacological agents, and their respective receptors and binding substances. (See e.g., Langan et al. eds., Ligand Assay, pp. 211 et seq., Masson Publishing U.S.A. Inc., New York, 1981).

As used herein, “the target cell or virus, if present in the sample, is allowed to bind to the magnetic microbead nonspecifically or with low specificity to form the conjugate” means that there is no specific binding between the magnetic microbead and the target cell or virus. For example, the binding between the magnetic microbead and the target cell, cellular organelle or virus is not mediated by a specific interaction between complementary biomolecules, such an interaction between ligand and receptor, antigen and antibody, substrate and enzyme, carbohydrate and lectin, and complementary nucleic acids, etc. It also means that the magnetic microbead does not comprise a moiety that can form a specific binding pair with the target cell or virus. For example, the moiety that is not comprised in the magnetic microbead is a biomolecule such as an amino acid, a peptide, a protein, a nucleoside, a nucleotide, an oligonucleotide, a nucleic acid, a vitamin, a monosaccharide, an oligosaccharide, a carbohydrate, a lipid and a complex thereof. Preferably, the moiety that is not comprised in the magnetic microbead is an antibody that specifically binds to the target cell or virus.

As used herein, “the magnetic microbead is modified to comprise a moiety that specifically binds to the target cell or virus” means that the magnetic microbead is modified to comprise a moiety that forms a specific binding pair with a target cell or virus.

As used herein, “the target cell or virus, if present in the sample, is allowed to bind to the magnetic microbead with high specificity” means that the magnetic microbead binds specifically with a target cell or virus.

As used herein, “antibody” refers to specific types of immunoglobulin, i.e., IgA, IgD, IgE, IgG, e.g., IgG₁, IgG₂, IgG₃, and IgG₄, and IgM. An antibody can exist in any suitable form and also encompass any suitable fragments or derivatives. Exemplary antibodies include a polyclonal antibody, a monoclonal antibody, a Fab fragment, a Fab′ fragment, a F(ab)₂ fragment, a Fv fragment, a diabody, a single-chain antibody and a multi-specific antibody formed from antibody fragments.

As used herein, “plant” refers to any of various photosynthetic, eucaryotic multi-cellular organisms of the kingdom Plantae, characteristically producing embryos, containing chloroplasts, having cellulose cell walls and lacking locomotion.

As used herein, “animal” refers to a multi-cellular organism of the kingdom of Animalia, characterized by a capacity for locomotion, nonphotosynthetic metabolism, pronounced response to stimuli, restricted growth and fixed bodily structure. Non-limiting examples of animals include birds such as chickens, vertebrates such fish and mammals such as mice, rats, rabbits, cats, dogs, pigs, cows, ox, sheep, goats, horses, monkeys and other non-human primates.

As used herein, “bacteria” refers to small prokaryotic organisms (linear dimensions of around 1 micron) with non-compartmentalized circular DNA and ribosomes of about 70S. Bacteria protein synthesis differs from that of eukaryotes. Many anti-bacterial antibiotics interfere with bacteria proteins synthesis but do not affect the infected host.

As used herein, “eubacteria” refers to a major subdivision of the bacteria except the archaebacteria. Most Gram-positive bacteria, cyanobacteria, mycoplasmas, enterobacteria, pseudomonas and chloroplasts are eubacteria. The cytoplasmic membrane of eubacteria contains ester-linked lipids; there is peptidoglycan in the cell wall (if present); and no introns have been discovered in eubacteria.

As used herein, “archaebacteria” refers to a major subdivision of the bacteria except the eubacteria. There are three main orders of archaebacteria: extreme halophiles, methanogens and sulphur-dependent extreme thermophiles. Archaebacteria differs from eubacteria in ribosomal structure, the possession (in some case) of introns, and other features including membrane composition.

As used herein, “fungus” refers to a division of eucaryotic organisms that grow in irregular masses, without roots, stems, or leaves, and are devoid of chlorophyll or other pigments capable of photosynthesis. Each organism (thallus) is unicellular to filamentous, and possesses branched somatic structures (hyphae) surrounded by cell walls containing glucan or chitin or both, and containing true nuclei.

As used herein, “virus” refers to an obligate intracellular parasite of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. The majority of viruses are recognized by the diseases they cause in plants, animals and prokaryotes. Viruses of prokaryotes are known as bacteriophages.

As used herein, “tissue” refers to a collection of similar cells and the intracellular substances surrounding them. There are four basic tissues in the body: 1) epithelium; 2) connective tissues, including blood, bone, and cartilage; 3) muscle tissue; and 4) nerve tissue.

As used herein, “organ” refers to any part of the body exercising a specific function, as of respiration, secretion or digestion.

As used herein, “sample” refers to anything which may contain a target cell, or virus that contains a target nucleic acid to be amplified using the present methods and/or kits. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood. Samples used for the present invention include such target-cell enriched cell preparation.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein, “magnetic substance” refers to any substance that has the properties of a magnet, pertaining to a magnet or to magnetism, producing, caused by, or operating by means of, magnetism.

As used herein, “magnetizable substance” refers to any substance that has the property of being interacted with the field of a magnet, and hence, when suspended or placed freely in a magnetic field, of inducing magnetization and producing a magnetic moment. Examples of magnetizable substances include, but are not limited to, paramagnetic, ferromagnetic and ferrimagnetic substances.

As used herein, “paramagnetic substance” refers to the substances where the individual atoms, ions or molecules possess a permanent magnetic dipole moment. In the absence of an external magnetic field, the atomic dipoles point in random directions and there is no resultant magnetization of the substances as a whole in any direction. This random orientation is the result of thermal agitation within the substance. When an external magnetic field is applied, the atomic dipoles tend to orient themselves parallel to the field, since this is the state of lower energy than antiparallel position. This gives a net magnetization parallel to the field and a positive contribution to the susceptibility. Further details on “paramagnetic substance” or “paramagnetism” can be found in various literatures, e.g., at Page 169-page 171, Chapter 6, in “Electricity and Magnetism” by B. I Bleaney and B. Bleaney, Oxford, 1975.

As used herein, “ferromagnetic substance” refers to the substances that are distinguished by very large (positive) values of susceptibility, and are dependent on the applied magnetic field strength. In addition, ferromagnetic substances may possess a magnetic moment even in the absence of the applied magnetic field, and the retention of magnetization in zero field is known as “remanence”. Further details on “ferromagnetic substance” or “ferromagnetism” can be found in various literatures, e.g., at Page 171-page 174, Chapter 6, in “Electricity and Magnetism” by B. I Bleaney and B. Bleaney, Oxford, 1975.

As used herein, “ferrimagnetic substance” refers to the substances that show spontaneous magnetization, remanence, and other properties similar to ordinary ferromagnetic materials, but the spontaneous moment does not correspond to the value expected for full parallel alignment of the (magnetic) dipoles in the substance. Further details on “ferrimagnetic substance” or “ferrimagnetism” can be found in various literatures, e.g., at Page 519-524, Chapter 16, in “Electricity and Magnetism” by B. I Bleaney and B. Bleaney, Oxford, 1975.

As used herein, “metal oxide particle” refers to any oxide of a metal in a particle form. Certain metal oxide particles have paramagnetic or super-paramagnetic properties. “Paramagnetic particle” is defined as a particle which is susceptible to the application of external magnetic fields, yet is unable to maintain a permanent magnetic domain. In other words, “paramagnetic particle” may also be defined as a particle that is made from or made of “paramagnetic substances”. Non-limiting examples of paramagnetic particles include certain metal oxide particles, e.g., Fe₃O₄ particles, metal alloy particles, e.g., CoTaZr particles.

As used herein, “poisonous agent” refers to any substance that is harmful to human health, e.g., chloroform or phenol.

B. Processes and Kits for Amplifying a Nucleic Acid of a Target Cell or Virus

In one aspect, the present invention is directed to a process for amplifying a nucleic acid of a target cell or virus, which process comprises: a) contacting a sample containing or suspected of containing a target cell or virus with a magnetic microbead; b) allowing said target cell or virus, if present in said sample, to bind to said magnetic microbead to form a conjugate between said target cell or virus and said magnetic microbead; and c) separating said conjugate from other undesirable constituents via a magnetic force to isolate said target cell or virus from said sample; and d) applying said separated conjugate to a nucleic acid amplification system to amplify a nucleic acid from said target cell or virus.

In another aspect, the present invention is directed to a kit for amplifying a nucleic acid of a target cell or virus, which kit comprises in a same or different container(s): a) a magnetic microbead for contacting a sample containing or suspected of containing a target cell or virus; b) means for allowing said target cell or virus, if present in said sample, to bind to said magnetic microbead to form a conjugate between said target cell or virus and said magnetic microbead; c) means for separating said conjugate from other undesirable constituents via a magnetic force from said sample; and d) a nucleic acid amplification system to amplify a nucleic acid from said target cell or virus. The kit can further comprise an instruction for using the kit for amplifying a nucleic acid of a target cell or virus from a sample.

The present processes and kits can be used to amplify any suitable target nucleic acid in any suitable target cell, cellular organelle or virus from any suitable sample. Exemplary samples include a clinical sample, serum, plasma, whole blood, sputum, cerebral spinal fluid, amniotic fluid, urine, gastrointestinal contents, hair, saliva, sweat, gum scrapings, marrow, tissue and cell culture. Exemplary target cells include animal cells, plant cells, fungus cells, bacterium cells, recombinant cells and cultured cells. Exemplary target viruses include eucaryotic cell viruses and bacteriophages. Target nucleic acids can be DNA, RNA or a mixture of combination thereof.

The magnetic microbeads can be prepared by any suitable methods. For example, the methods disclosed in CN 01/109870.8 or WO02/075309 can be used. Any suitable magnetizable substance can be used to prepare the magnetic microbeads useful in the present processes and kits. No-limiting examples of the magnetizable substances include ferrimagnetic substance, ferromagnetic substance, paramagnetic substance or superparamagnetic substances. In a specific embodiment, the magnetic microbeads comprise a paramagnetic substance, e.g., a paramagnetic metal oxide composition. Preferably, the paramagnetic metal oxide composition is a transition metal oxide or an alloy thereof. Any suitable transition metals can be used, such as iron, nickel, copper, cobalt, manganese, tantalum (Ta), zinc and zirconium (Zr). In a preferred embodiment, the metal oxide composition is Fe₃O₄ or Fe₂O₃. In another example, the magnetizable substance used in the magnetic microbeads comprises a metal composition. Preferably, the metal composition is a transition metal composition or an alloy thereof such as iron, nickel, copper, cobalt, manganese, tantalum, zirconium and cobalt-tantalum-zirconium (CoTaZr) alloy.

The magnetic microbeads may be prepared from the available primary beads, from raw materials or from metal oxides that are encapsulated by monomers which when crosslinked form rigid, polymeric coatings as disclosed in U.S. Pat. No. 5,834,121. As used herein, “rigid” refers to a polymeric coating that is cross linked to the extent that the polymeric coating stabilizes the metal oxide particle within the coating (i.e. the coating essentially does not swell or dissolve) so that the particle remains enclosed therein. As used herein, “microporous” refers to a resinous polymeric matrix that swells or expands in polar organic solvent. As used herein, “load” is used to mean the capacity of the bead for attachment sites useful for functionalization or derivatization.

Suitable substances which may be incorporated as magnetizable materials, for example, include iron oxides such as magnetite, ferrites of manganese, cobalt, and nickel, hematite and various alloys. Magnetite is the preferred metal oxide. Frequently, metal salts are taught to be converted to metal oxides then either coated with a polymer or adsorbed into a bead comprising a thermoplastic polymer resin having reducing groups thereon. When starting with metal oxide particles to obtain a hydrophobic primary bead, it is necessary to provide a rigid coating of a thermoplastic polymer derived from vinyl monomers, preferably a cross-linked polystyrene that is capable of binding or being bound by a microporous matrix. Magnetic particles may be formed by methods known in the art, e.g., procedures shown in Vandenberge et al., J. of Magnetism and Magnetic Materials, 15-18:1117-18 (1980); Matijevic, Acc. Chem. Res., 14:22-29 (1981); and U.S. Pat. Nos. 5,091,206; 4,774,265; 4,554,088; and 4,421,660. Examples of primary beads that may be used in this invention are shown in U.S. Pat. Nos. 5,395,688; 5,318,797; 5,283,079; 5,232,7892; 5,091,206; 4,965,007; 4,774,265; 4,654,267; 4,490,436; 4,336,173; and 4,421,660. Or, primary beads may be obtained commercially from available hydrophobic or hydrophilic beads that meet the starting requirements of size, sufficient stability of the polymeric coating to swell in solvents to retain the paramagnetic particle, and ability to adsorb or absorb the vinyl monomer used to form the enmeshing matrix network. Preferably, the primary bead is a hydrophobic, polystyrene encapsulated, paramagnetic bead. Such polystyrene paramagnetic beads are available from Dynal, Inc. (Lake Success, N.Y.), Rhone Poulonc (France), and SINTEF (Trondheim, Norway). The use of toner particles or of magnetic particles having a first coating of an unstable polymer which are further encapsulated to produce an exterior rigid polymeric coating is also contemplated.

The magnetic microbeads used in the present processes and kits can have any suitable size, e.g., having a diameter ranging from about 5 to about 50,000 nanometers.

The magnetic microbeads used In the present processes and kits can be untreated or can be modified, e.g., modified with an organic molecule. In a specific embodiment, the magnetic microbead is modified to comprise a hydroxyl, a carboxyl or an epoxy group. In another specific embodiment, the magnetic microbead is modified to comprise a moiety, e.g., an antibody or functional fragment thereof, that specifically binds to the target cell or virus. Alternatively, the target cell or virus, if present in the sample, can be allowed to bind to the magnetic microbead with high specificity to form the conjugate. Also alternatively, the target cell or virus, if present in the sample, can be allowed to bind to the magnetic microbead nonspecifically or with low specificity to form the conjugate.

The separated conjugate formed between the target cell or virus and the magnetic microbead can be used directly in amplifying the target nucleic acid contained therein. Alternatively, the process can further comprise washing the separated conjugate to remove the undesirable constituents before applying separated conjugate to a nucleic acid amplification system.

The present processes can be performed manually. Preferably, the present processes are automated. Any, some or all steps of the present processes can be automated. For example, the sample contacting, binding, separating, as well as any other additional steps such as washing, target cell or virus releasing, and biological material recovering or amplifying step(s) can be automated.

The present processes can be performed within any suitable time frame. For example, the present processes can be performed within a time ranging from about 0.5 minute to about 30 minutes.

The present processes can be performed at any suitable temperature. For example, the present processes can be performed at an ambient temperature ranging from about 0° C. to about 35° C. without temperature control.

The present processes can be performed in any suitable volume. For example, the present processes can be performed in a volume ranging from about 5 μl to about 50 μl.

The present processes can be performed in an eppendorf tube. The present processes can be performed in the absence of a precipitation or centrifugation procedure. The present processes can be performed in the absence of a poisonous agent.

In one specific embodiment, the target cell is a leukocyte isolated from whole blood, marrow or lympha, e.g., fresh or low-temperature conserved whole blood, marrow or lympha In another specific embodiment, the target cell is an epithelia cast-off cell or a bacteria cell isolated from saliva, urine and tissue culture as stated in China Patent Application No. 02153992.8.

Any suitable nucleic acid amplification system can be used in the present processes and kits. Exemplary nucleic acid amplification systems include polymerase chain reaction (PCR) (U.S. Pat. Nos. 4,683,195 and 4,683,202 and Ausubel (Ed.) Current Protocols in Molecular Biology, 15. The Polymerase Chain Reaction, John Wiley & Sons, Inc. (2000)), ligase chain reaction (LCR), nucleic acid sequence-based amplification (NASBA) (U.S. Pat. Nos. 5,409,818 and 5,554,517), strand displacement amplification (SDA) and transcription-medicated amplification (TMA) systems.

The process can further comprise removing cells from a sample containing or suspected of containing a target virus or bacteriophage before contacting the sample with a magnetic microbead.

In still another aspect, the present invention is directed to a process for amplifying a nucleic acid of a target cell or virus, which process comprises: a) contacting a sample containing or suspected of containing a target cell or virus with a magnetic microbead; b) allowing said target cell or virus, if present in said sample, to bind to said magnetic microbead to form a conjugate between said target cell or virus and said magnetic microbead; and c) separating said conjugate from other undesirable constituents via a magnetic force to isolate said target cell or virus from said sample; d) releasing a nucleic acid from said cell-microbead or virus-microbead conjugate to form a nucleic acid-microbead conjugate; and d) applying said nucleic acid-microbead conjugate to a nucleic acid amplification system to amplify said nucleic acid from said target cell or virus.

The process can further comprise washing the nucleic acid-microbead conjugate to remove the undesirable constituents before applying the nucleic acid-microbead conjugate to a nucleic acid amplification system. The process can further comprise separating nucleic acid-microbead conjugate from other undesirable constituents via a magnetic force before applying the nucleic acid-microbead conjugate to a nucleic acid amplification system.

In yet another aspect, the present invention is directed to a kit for amplifying a nucleic acid of a target cell or virus, which kit comprises in a same or different container(s): a) a magnetic microbead for contacting a sample containing or suspected of containing a target cell or virus; b) means for allowing said target cell or virus, if present in said sample, to bind to said magnetic microbead to form a conjugate between said target cell or virus and said magnetic microbead; c) means for separating said conjugate from other undesirable constituents via a magnetic force from said sample; d) means for releasing a nucleic acid from said cell-microbead or virus-microbead conjugate to form a nucleic acid-microbead conjugate; and e) a nucleic acid amplification system to amplify a nucleic acid from said target cell or virus.

The kit can further comprise an instruction for using the kit for amplifying a nucleic acid of a target cell or virus from a sample.

C. Exemplary Embodiments

The embodiments described herein relate generally to the preparation of PCR templates and the process for nucleic acid, e.g., gene, amplification using magnetic micro-beads. Through the nonspecific or low-specificity adsorption to cells and nucleic acids, the cells and biological materials (such as leukocyte, virus, epithelial cell and cultured cell) containing target biological molecules (such as nucleic acid and protein) can be separated from the whole blood, plasma, serum, marrow, saliva, urine and culture solution of cells and tissues and they together with the magnetic micro-beads form the micro-beads-cells conjugates. After the lysis of the eluted cells, the discharged nucleic acids are absorbed by the magnetic micro-beads and they form the micro-beads-nucleic-acid conjugates. The micro-beads-cells and micro-beads-nucleic-acid conjugates are added into the PCR system as the templates for the PCR reaction. Because of the removal of the impurity and PCR inhibitor and the insignificant influence of the magnetic micro-beads, the sensitivity and stability of gene amplification is not affected.

An important aspect of the embodiments is to separate target cells using the magnetic micro-beads and that the obtained cells can be used as the PCR templates of the target gene. Most of the inhibiting factors can be eliminated. The magnetic micro-beads can adsorb the cells or virus and nucleic acids. This simple and rapid process can be used in diagnostic analysis and research and it is easy to build up an automatic and micromatic device.

The embodiments have the following main advantages:

(1) The volume of samples and agents used in this process is small. This process can dispose 5 μl of whole blood or saliva and the volume of the used agents is less than 50 μl.

(2) The preparation process can be operated at room temperature without centrifugation and temperature control.

(3) The operation is simple, rapid and convenient. The whole process takes only about 0.5-15 minutes.

(4) The transfer operation in the process, as well as the possibility of contaminating the samples, can be avoided or decreased.

(5) The separation method is universal and is suitable for many kinds of samples.

(6) There is no hazard to the operator and environment during this process.

(7) It is easy to automate and miniaturize the process to have a concurrent template preparation and amplifications process.

1. Nonspecific Adsorption and Separation of Cells and Preparation of PCR Templates

It is a pivotal and basic step to separate cells from the samples in biological science. The density gradient centrifugation is often used based on the size and density difference of the cells. However, the requirement of centrifugation makes it difficulty to build up the mini devices. The barrier device is built on the chip to filter the target cells based on the size difference of the cells. This device is not universal because of its difficult machining. In principle, there are two kinds of methods to separate cells using the magnetic micro-beads. One is the specific separation of cells using the magnetic micro-beads derived with a specific antibody. The other is to separate the cells by using the selective centrifugation or the adsorption difference for the cells of the magnetic micro-beads. The first method is suitable for many kinds of cells and the separated cells have high specificity. But the magnetic micro-beads are not only expensive but also require rigorous transportation and preservation conditions and lose their biological activity easily. The second method has low universality. But the magnetic micro-beads not only are cheap but also don't require rigorous transportation and preservation conditions. The environmental conditions have little influence on the separation performance and the method is simple.

In this embodiment, the magnetic micro-beads uncoated or coated with organic materials can enrich the target cells effectively and adsorb the nucleic acids under the appropriate chemical and physical circumstance. The obtained micro-beads-cells and micro-beads-nucleic-acid conjugates can be used as the PCR templates for gene amplification. Thus the template preparation and the gene amplification are integrated and are suitable for the build-up of a PCR device. The process is simple and rapid. It only takes one minute to prepare the templates from the whole blood, plasma, serum, marrow, saliva, urine and culture solution of cells and tissues.

1.1 Preparation of the Solid Carrier

The preparation of the coated magnetic micro-beads can be found in CN 01/109870.8 or WO02/075309.

1.2 Operation Program

(1) Small magnetic micro-beads (suspended in Tris-EDTA buffer, pH 6.0) are added into a liquid biological sample. The mixture is agitated gently by vortexing and incubated at room temperature for 3 min to form the micro-beads-cells conjugates.

(2) The magnetic micro-beads-cells conjugates are separated by a magnetic field and the supernatant is discarded. The magnetic micro-bead-cells conjugates are washed once with 70% ethanol solution. The washed micro-beads-cells conjugates can be added into a PCR system directly for gene amplification.

(3) Small cell lysis solution is added into the mixture and the suspension is mixed uniformly by vortexing and incubated at room temperature for 1 min to lyse the cells. Isopropyl alcohol can be added into the mixture and the suspension is mixed uniformly by vortexing, then allow to stand still for 5 min to form the conjugates.

(4) The magnetic micro-beads-nucleic-acids conjugates are separated by the magnetic field and the supernatant is discarded. The magnetic micro-beads-nucleic-acids conjugates are washed twice with 70% ethanol solution to eliminate the salt. The washed micro-beads-nucleic-acids conjugates can be directly added into the PCR system for gene amplification.

1.3 Chemical Agents Content

(1) TE buffer (pH 6.0): 10 mM EDTA/25 mM Tris-HCl. Tris-EDTA (pH 6.0): 10 mM EDTA/25 mM Tris-HCl.

(2) lysis solution: NaI 11.25 g; Urea 12.0 g; Triton X-100 0.65 ml; TE (pH 8.0) 30 ml: 10 mM EDTA/25 mM Tris-HCl.

1.4. Main Advantages

This method has the following main advantages: (1) simple and rapid operation, which takes only about 1-10 min; (2) requiring only an eppendorf tube, without precipitation; (3) the obtained products suitable for subsequent biological operations; (4) easy to realize the automatic operation; (5) safe operation without poisonous using agent; (6) operation at room temperature; (7) easy preservation of the magnetic micro-beads, which have insignificant influence on the separation effect.

2. Specific Adsorption and Separation Technique of Cells and PCR Template Preparation

2.1. Operation Program

(1) Small magnetic micro-beads derived with an antibody reacting with an antigen which is on the surface of specific cells are added into a liquid biological sample. The mixture is agitated gently by vortexing and incubated at room temperature for 15 min to form the micro-beads-cells conjugates.

(2) The magnetic micro-beads-cells conjugates are separated by a magnetic field and the supernatant is discarded. The magnetic micro-bead-cells conjugates are washed once with 70% ethanol solution. The washed micro-beads-cells conjugates can be directly added into a PCR system for gene amplification.

(3) Small cell lysis solution is added into the mixture and the suspension is mixed uniformly by vortexing and incubated at room temperature for 1 min to lyse the cells. Isopropyl alcohol can be added into the mixture and the suspension is mixed uniformly by vortexing, then allow to stand still for 5 min to form the conjugates.

(4) The magnetic micro-beads-nucleic-acids conjugates are separated by the magnetic field and the supernatant is discarded. The magnetic micro-beads-nucleic-acids conjugates are washed once with 70% ethanol solution to eliminate the salt. The washed micro-beads-nucleic-acids conjugates can be directly added into a PCR system for gene amplification.

2.2. Chemical Agents Content

(1) TE buffer (pH 6.0): 10 mM EDTA/25 mM Tris-HCl; Tris-EDTA (pH 6.0): 10 mM EDTA/25 mM Tris-HCl.

(2) Lysis solution: NaI 11.25 g; Urea 12.0 g; Triton X-100 0.65 ml; TE (pH 8.0) 30 ml: 10 mM EDTA/25 mM Tris-HCl.

2.3. Main Advantages

This method has the following main advantages: (1) simple and rapid operation, which takes only 20-30 min; (2) requiring only an eppendorf tube, without precipitation; (3) the obtained products suitable for subsequent biological operations; (4) easy to realize the automatic operation; (5) safe operation without using poisonous agents; (6) easy elimination of the PCR inhibitor.

### D. EXAMPLES

### Example 1

### Template Preparation and Amplification of HLA-A Gene from Human Whole Blood

Human whole blood from healthy donors was anticoagulated with ACD with ⅙ volume of the blood. The procedure of isolation of leukocytes is as follows. To a 1.5 mL

Eppendorf tube containing 10 μL of 15 μg/μL magnetic micro-beads suspended in Tris-EDTA buffer (pH 6.0) were added 50 μL anticoagulated blood. The mixture was agitated gently by vortexing for 15 s and incubated at room temperature for 3 min. Then the micro-beads-leukocytes conjugates were immobilized on a magnetic stand and the supernatant was discarded. The magnetic micro-beads-leukocytes conjugates were washed twice with 100 μL 70% ethanol solution. The above washed micro-beads-leukocytes conjugates were directly added into a PCR system for HLA-A gene amplification. The products were analyzed by agarose gel electrophoresis.

The above micro-beads-leukocytes conjugates can be used to extract the nucleic acids. Fifty (50) μL cell lysis solution (NaI 11.25 g; Urea 12.0 g; Triton X-100 0.65 ml;

TE (pH 8.0) 30 ml: 10 mM EDTA/25 mM Tris-HCl) were added into the mixture and the suspension was mixed uniformly by vortexing and incubated at room temperature for min to lyse the leukocytes. Three hundred (300) μL isopropyl alcohol were added into the mixture and the suspension was mixed uniformly by vortexing, and then let stand still for 5 min. The magnetic micro-bead-nucleic-acids conjugates were immobilized on a magnetic stand and the supernatant was discarded. The magnetic micro-bead-nucleic-acids conjugates were washed twice with 100 μL 70% ethanol solution. After thoroughly evaporating ethanol under room temperature, 50 μL solution of Tris-EDTA (pH 6.0) was added into the conjugates and it was incubated at room temperature for 10 min to elute DNA. The magnetic micro-bead-nucleic-acids conjugates were added into the PCR system to amplify the HLA-A gene. The eluted DNA can be used as the template for gene amplification.

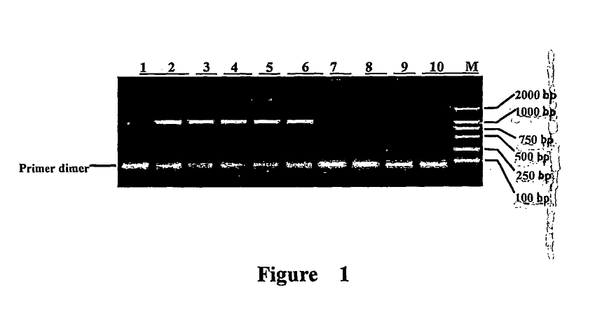

The above three templates have little difference on the efficiency and sensitivity of gene amplification, which is shown in FIG. 1.

### Example 2

### Template Preparation and Amplification of HLA-A Gene from Saliva

The used saliva was offered by healthy donors. The procedure of isolation of leukocytes is as follows. To a 1.5 mL Eppendorf™ tube containing 10 μL of 15 μg/μL magnetic micro-beads suspended in Tris-EDTA buffer (pH 6.0) were added 50 μL saliva. The mixture was agitated gently by vortexing for 15 s and incubated at room temperature for 3 min. Then the micro-beads-epithelial cells conjugates were immobilized on a magnetic stand and the supernatant was discarded. The magnetic micro-beads-epithelial-cells conjugates were washed twice with 100 μL 70% ethanol solution. The above washed micro-beads-epithelial-cells conjugates were directly added into a PCR system for HLA-A gene amplification. The products were analyzed by agarose gel electrophoresis. The yield and purity of the amplified products using the template prepared by the magnetic micro-beads was compared to them using the template prepared by the eluted DNA.

### Example 3

### Template Preparation and Amplification of HLA-A Gene from Human Whole Blood

Human whole blood from healthy donors was anticoagulated with ACD with ⅙ volume of the blood. The procedure of isolation of leukocytes is as follows. To a 1.5 mL Eppendorf™ tube containing 10 μL of 15 μg/μL magnetic micro-beads suspended in Tris-EDTA buffer (pH 6.0) were added 50 μL anticoagulated blood mixed with 100 μL cell lysis solution (0.5% Na₂EDTA, 0.1M Tris, 0.1M NaCl, 1% NP-40, 30 μl proteinase K (20 mg/mL, pH 7.8)). The suspension was mixed uniformly by vortexing and incubated at room temperature for 15 min. The magnetic micro-bead-DNA-anti-DNA conjugates were immobilized on a magnetic stand and the supernatant was discarded. Fifth (50) μL solution of Tris-EDTA (pH 6.0) was added into the conjugates and it was incubated at room temperature for 10 min to elute DNA. The eluted DNA was added into a PCR system to amplify the HLA-A gene. The products were analyzed directly by agarose gel electrophoresis and UV spectroscopy. This method, without using poisonous agents, has excellent specificity and high separation efficiency.

### Example 4

### Template Preparation and Amplification of HBV Virus Gene from Human Whole Blood

Human whole blood from donors carrying HBV virus was anticoagulated with ACD with ⅙ volume of the blood. The procedure of isolation of virus is as follows. Two hundred (200) μl serum were separated from 500 μl whole blood. It was added into the Tris-EDTA buffer (pH 6.0) containing 50 μL of 15 μg/μL magnetic micro-beads derived antibody anti HBV virus. The suspension was mixed uniformly by gentle vortexing and incubated at room temperature for 15 min. The magnetic micro-bead-virus-antibody anti HBV virus conjugates were immobilized on a magnetic stand and the supernatant was discarded. The conjugates were added into a PCR system to amplify the HBV gene. The products were analyzed directly by agarose gel electrophoresis and UV spectroscopy. This method, without using poisonous agents, has excellent specificity and high separation efficiency.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

What is claimed is:

1. A process for amplifying a nucleic acid of a target cell or virus, which process comprises:
a) contacting a sample containing or suspected of containing a target cell or virus with a magnetic microbead;
b) allowing said target cell or virus, if present in said sample, to bind to said magnetic microbead to form a conjugate between said target cell or virus and said magnetic microbead; and
c) separating said conjugate from other undesirable constituents via a magnetic force to isolate said target cell or virus from said sample; and
d) applying said separated conjugate to a nucleic acid amplification system to amplify a nucleic acid from said target cell or virus.

2. The process of claim 1, wherein the sample is a clinical sample.

3. The process of claim 1, wherein the sample is selected from the group consisting of serum, plasma, whole blood, sputum, cerebral spinal fluid, amniotic fluid, urine, gastrointestinal contents, hair, saliva, sweat, gum scrapings, marrow, tissue and cell culture.

4. The process of claim 1, wherein the target cell is selected from the group consisting of an animal cell, a plant cell, a fungus cell, a bacterium cell, a recombinant cell and a cultured cell.

5. The process of claim 1, wherein the target virus is an eucaryotic cell virus or a bacteriophage.

6. The process of claim 1, wherein the magnetic microbead comprises a magnetizable substance selected from the group consisting of a paramagnetic substance, a ferromagnetic substance and a ferrimagentic substance.

7. The process of claim 6, wherein the magnetizable substance comprises a metal composition.

8. The process of claim 7, wherein the metal composition is a transition metal composition or an alloy thereof.

9. The process of claim 8, wherein the transition metal is selected from the group consisting of iron, nickel, copper, cobalt, manganese, tantalum, zirconium and cobalt-tantalum-zirconium (CoTaZr) alloy.

10. The process of claim 7, wherein the metal composition is Fe₃O₄.

11. The process of claim 1, wherein the magnetic microbead has a diameter ranging from about 5 to about 50,000 nanometers.

12. The process of claim 1, wherein the magnetic microbead is untreated or modified with an organic molecule.

13. The process of claim 1, wherein the magnetic microbead is modified to comprise a hydroxyl, a carboxyl or an epoxy group.

14. The process of claim 1, wherein the magnetic microbead is modified to comprise a moiety that specifically binds to the target cell or virus.

15. The process of claim 14, wherein the moiety is an antibody or functional fragment thereof.

16. The process of claim 1, wherein the target cell or virus, if present in the sample, is allowed to bind to the magnetic microbead nonspecifically or with low specificity to form the conjugate.

17. The process of claim 1, wherein the target cell or virus, if present in the sample, is allowed to bind to the magnetic microbead with high specificity to form the conjugate.

18. The process of claim 1, which further comprises washing the separated conjugate to remove the undesirable constituents before applying separated conjugate to a nucleic acid amplification system.

19. The process of claim 1, which is automated.

20. The process of claim 1, which is completed within a time ranging from about 0.5 minute to about 30 minutes.

21. The process of claim 1, which is conducted in an eppendorf tube.

22. The process of claim 1, which is conducted in the absence of a precipitation or centrifugation procedure.

23. The process of claim 1, which is conducted in the absence of a poisonous agent.

24. The process of claim 1, which is conducted at an ambient temperature ranging from about 0° C. to about 35° C. without temperature control.

25. The process of claim 1, wherein the sample volume ranges from about 5 μl to about 50 μl.

26. The process of claim 1, wherein the target cell is a leukocyte isolated from whole blood, marrow or lympha.

27. The process of claim 1, wherein the target cell is an epithelia cast-off cell or a bacteria cell isolated from saliva, urine and tissue culture.

28. The process of claim 1, wherein the nucleic acid amplification system is selected from the group consisting of polymerase chain reaction (PCR), ligase chain reaction (LCR), nucleic acid sequence-based amplification (NASBA), strand displacement amplification (SDA) and transcription-medicated amplification (TMA).

29. The process of claim 1, which further comprises removing cells from a sample containing or suspected of containing a target virus or bacteriophage before contacting the sample with a magnetic microbead.

30. A kit for amplifying a nucleic acid of a target cell or virus, which kit comprises in a same or different container(s):
a) a magnetic microbead for contacting a sample containing or suspected of containing a target cell or virus;
b) means for allowing said target cell or virus, if present in said sample, to bind to said magnetic microbead to form a conjugate between said target cell or virus and said magnetic microbead;
c) means for separating said conjugate from other undesirable constituents via a magnetic force from said sample; and
d) a nucleic acid amplification system to amplify a nucleic acid from said target cell or virus.

31. The kit of claim 30, which further comprises an instruction for using the kit for amplifying a nucleic acid of a target cell or virus from a sample.

32. A process for amplifying a nucleic acid of a target cell or virus, which process comprises:
a) contacting a sample containing or suspected of containing a target cell or virus with a magnetic microbead;
b) allowing said target cell or virus, if present in said sample, to bind to said magnetic microbead to form a conjugate between said target cell or virus and said magnetic microbead; and
c) separating said conjugate from other undesirable constituents via a magnetic force to isolate said target cell or virus from said sample;
d) releasing a nucleic acid from said cell-microbead or virus-microbead conjugate to form a nucleic acid-microbead conjugate; and
d) applying said nucleic acid-microbead conjugate to a nucleic acid amplification system to amplify said nucleic acid from said target cell or virus.

33. The process of claim 32, which further comprises washing the nucleic acid-microbead conjugate to remove the undesirable constituents before applying the nucleic acid-microbead conjugate to a nucleic acid amplification system.

34. The process of claim 32, which further comprises separating nucleic acid-microbead conjugate from other undesirable constituents via a magnetic force before applying the nucleic acid-microbead conjugate to a nucleic acid amplification system.

35. A kit for amplifying a nucleic acid of a target cell or virus, which kit comprises in a same or different container(s):
a) a magnetic microbead for contacting a sample containing or suspected of containing a target cell or virus;
b) means for allowing said target cell or virus, if present in said sample, to bind to said magnetic microbead to form a conjugate between said target cell or virus and said magnetic microbead;
c) means for separating said conjugate from other undesirable constituents via a magnetic force from said sample;
d) means for releasing a nucleic acid from said cell-microbead or virus-microbead conjugate to form a nucleic acid-microbead conjugate; and
e) a nucleic acid amplification system to amplify a nucleic acid from said target cell or virus.

36. The kit of claim 35, which further comprises an instruction for using the kit for amplifying a nucleic acid of a target cell or virus from a sample.
